(19) United States
(12) Patent Application Publication
(10) Pub. No.: US 2022/0026381 A1
(43) Pub. Date: Jan. 27, 2022

(54) METHOD FOR DETERMINING THE SENSITIVITY TO OXIDATION OF A BITUMINOUS BINDER BY ELECTRON SPIN RESONANCE

(71) Applicants: TOTAL MARKETING SERVICES, PUTEAUX (FR); Centre national de la recherche scientifique, PARIS (FR); Université de Lille, LILLE (FR)

(72) Inventors: Manuel MERCE, BÉNESSE-MAREMNE (FR); Simon PONDAVEN, LYON (FR); Philippe MARCHAND, COMMUNAY (FR); Hervé VEZIN, VILLENEUVE D'ASCQ (FR); Karima BEN TAYEB MEZIANE, VILLENEUVE D'ASCQ (FR)

(73) Assignees: TOTAL MARKETING SERVICES, PUTEAUX (FR); Centre national de la recherche scientifique, PARIS (FR); Université de Lille, LILLE (FR)

(21) Appl. No.: 17/297,128

(22) PCT Filed: Nov. 26, 2019
(86) PCT No.: PCT/EP2019/082585

§ 371 Date: May 26, 2021

(30) Foreign Application Priority Data
Nov. 29, 2018 (FR) 1872084

(51) Int. Cl. G01N 24/10

(52) U.S. Cl. CPC G01N 24/10

(57) ABSTRACT

This invention concerns a method for analysing the ageing stability of a bituminous binder, in particular by reference to its susceptibility to oxidation, by analysing a sample of the bituminous binder by means of electron spin resonance and measuring the integral of the signal of the carbon-centred stable radicals; accelerated ageing of the bituminous binder, comprising: i) heating the bituminous binder, followed by ii) heating the bituminous binder resulting from step i) under pressure; analysing a sample of the aged bituminous binder obtained from step b) by means of electron spin resonance and measuring the integral of the signal of the carbon-centred stable radicals; and comparing the integral of the signal of the carbon-centred stable radicals of the bituminous binder obtained from the first step and that obtained from the previous step.

### CROSS REFERENCE TO RELATED APPLICATIONS

The present application is a U.S. National Phase Application under 35 U.S.C. § 371 of International Patent Application No. PCT/EP2019/082585, filed Nov. 26, 2019, which claims priority of French Patent Application No. 18 72084, filed Nov. 29, 2018. The entire contents of which are hereby incorporated by reference.

### FIELD OF THE INVENTION

This invention concerns the analysis of the ageing stability of a bituminous binder, in particular in terms of its susceptibility to oxidation, by means of electron spin resonance. This invention also concerns a method for classifying bituminous binders based on their susceptibility oxidation, a method for continuous monitoring of the production of a bituminous binder, and a method for quality control of a bituminous binder.

### BACKGROUND

The ageing of a bituminous binder is due, in particular, to the existence of oxidation mechanisms. This progressive ageing causes changes in the structure of the bituminous binder and an alteration of its macroscopic properties, e.g., making it more fragile and thus more susceptible to the formation of fissures.

Thus, there is an interest in being able to evaluate the propensity of a bituminous binder to resist ageing, in particular its oxidation resistance.

Buckmaster et al. (Advances in Chemistry Series, 1993, 229 (Magnetic Resonance of Carbonaceous Solids), 483-506) describes the determination of spectroscopic parameters of bituminous coals *subjected to oxidation at low temperature by means of ESR. Rossi et al. (Applied Sciences, 2018, 8, 1405-1419) describes the use of ESR to study the influence of the addition of natural antioxidants to bitumens in order to prevent ageing.

However, these methods do not concern the analysis of a raw bituminous binder, or they do not allow for measurement of the long-term impact of ageing.

Thus, there is an interest in developing a method for analyzing a bituminous binder that allows for a rapid, reliable determination of its ageing stability, in particular in terms of its susceptibility to oxidation.

### SUMMARY

One object of this invention is to provide a rapid, reliable method for analyzing the ageing stability of a bituminous binder, in particular by reference to its susceptibility to oxidation.

Another object of this invention is to provide a method that allows for a bituminous binder to be classified based on its ageing stability, and in particular its susceptibility to oxidation.

Thus, this invention concerns a method for analyzing the ageing stability of a bituminous binder, in particular by reference to its susceptibility to oxidation, comprising:
a) analyzing a sample of the bituminous binder by means of electron spin resonance and measuring the integral of the signal of the carbon-centered stable radicals;
b) accelerated ageing of the bituminous binder, comprising: i) heating the bituminous binder, followed by ii) heating the bituminous binder resulting from step i) under pressure;
c) analyzing a sample of the aged bituminous binder obtained from step b) by means of electron spin resonance and measuring the integral of the signal of the carbon-centered stable radicals;
d) comparing the integral of the signal of the carbon-centered stable radicals of the bituminous binder obtained from step a) and that obtained from step c).

The greater the value obtained in step d), the greater the susceptibility of the bituminous binder to oxidation, and, thus, to ageing.

In the context of this invention, the terms ‘bituminous binder’ and ‘bitumen’ are synonymous.

Examples of bitumens according to the invention especially include bitumens of natural origin, those contained in natural bitumen deposits, natural asphalt deposits, or bituminous sands, as well as bitumens originating from the refinement of crude oil.

Advantageously, the bitumens according to the invention are selected from the bitumens resulting from crude oil refinement. Bitumens may be selected from amongst bitumens or bitumen mixtures arising from crude oil refinement, in particular bitumens containing asphaltenes or tar.

Bitumens may be obtained by conventional methods for manufacturing bitumens in refineries, in particular by direct distillation or vacuum distillation of oil. These bitumens may in some cases be reduced in viscosity and/or deasphalted, and/or air-rectified in the manner known to persons skilled in the art. It is customary to vacuum-distil the atmospheric residues originating from the atmospheric distillation of crude oil. As such, this manufacturing method corresponds to a sequence of atmospheric distillation and vacuum distillation, with the load supplying the vacuum distillation corresponding to atmospheric residues. These vacuum residues originating in the vacuum distillation tower may also constitute a bitumen according to the invention. It is also customary to inject air into a load usually consisting of distillates and heavy oils originating from the vacuum distillation of atmospheric residues from oil distillation. This method allows an expanded, partially expanded, air-rectified, or partially air-rectified base to be obtained.

The various bitumens or bitumen bases obtained by refinement methods may be combined in order to obtain the best technical balance. The bitumen may also be a recycled bitumen. The bitumens may be hard- or soft-grade bitumens.

Advantageously, the bitumen is selected from amongst bitumens of natural origin, from amongst bitumens originating from oil refinement such as atmospheric distillation residues, vacuum distillation residues, reduced-viscosity residues, expanded residues, and mixtures and combinations thereof.

In the method according to the invention, the bituminous binder is analyzed in the form in which it is obtained from the refinement process. Preferably, the bituminous binder contains no additives such as antioxidants. Preferably, the bituminous binder has not been subjected to any extraction step using an organic solvent or a mixture of two or more organic solvents, e.g. aliphatic or aromatic hydrocarbons. Preferably, the bituminous binder is analyzed as a whole.

Amongst the known art analysis methods, electron spin resonance (ESR) is particularly well suited because it is relatively rapid and requires a sample of only a few milligrams or tens of milligrams.

There are ESR analysis methods that allow for a measurement of the development of a bituminous binder that has been subjected to oxidation.

In the context of this invention, ESR analysis consists of recording the ESR spectrum of a sample of a bituminous binder over a narrow field range (between 3400 and 3600 G) in order to concentrate on detecting the signal corresponding to the stable carbon-centered radicals present in the bituminous binder. This signal preferably ranges between 3400 and 3500 G.

In the context of this invention, a ‘carbon-centered’ stable radical is a radical carried on a carbon.

In the context of this invention, ‘measuring the integral of the signal of the carbon-centered stable radicals’ consists of processing the ESR signal corresponding to the stable carbon-centered radicals in order to obtain the value of the integral of this signal, with this integral value being normalized by the mass of the sample analyzed. According to the invention, the integral of the free radicals corresponds to the peak area corresponding to the carbon-centered stable radicals. The processing of the ESR signal is known to persons skilled in the art.

Preferably, the value of the integral of the ESR signal of the carbon-centered stable radicals of the bituminous binder does not include the vanadyl ions.

Preferably, in the method according to the invention, the step i) of accelerated ageing of the bituminous binder takes place between 100 and 200° C. for a duration of between 5 and 200 min, preferably between 150 and 190° C. over a duration of between 50 and 100 min, more preferably at 163° C. for 75 min.

Preferably, step i) corresponds to an RTFOT test (Rolling Thin Film Over Test). This test is governed by standard EN 12607-1.

Preferably, in the method according to the invention, the step ii) of accelerated ageing of the bituminous binder occurs between 50 and 150° C., at a pressure ranging from 0.5-5 MPa, over a duration of between 1 and 100 h, preferably between 70 and 120° C. at a pressure ranging between 1 and 3 MPa, over a duration of between 10 and 30 h, more preferably at 100° C., at a pressure of 2.1 MPa over a period of 20 h.

More preferably, step ii) consists of a long-term accelerated ageing test in a PAV (Pressure Ageing Vessel). This test is governed by standard EN 14769.

This invention also concerns a method for classifying a bituminous binder as a function of its ageing stability, in particular as a function of its susceptibility to oxidation, comprising:
1) analyzing a sample of the bituminous binder by means of electron spin resonance and measuring the integral of the signal of the carbon-centered stable radicals;
2) accelerated ageing of the bituminous binder;
3) analyzing a sample of the aged bituminous binder obtained from step 2) by means of electron spin resonance and measuring the integral of the signal of the carbon-centered stable radicals;
4) determining the percent increase in the number of carbon-centered stable radicals in the bituminous binder by comparing the integral of the signal of the carbon-centered stable radicals of the bituminous binder obtained in step 1) and that obtained in step 3);
5) determining the level of susceptibility of the bituminous binder:
   low in the case of an increase in the number of stable radicals of less than 60%;
   medium in the case of an increase in the number of stable radicals of 60-80%, or
   high in the case of an increase in the number of stable radicals of more than 80%.

In the context of this invention, ‘accelerated ageing of the bituminous binder’ according to the aforementioned classification method corresponds to: i) heating the bituminous binder, followed by ii) heating the bituminous binder obtained from step i) under pressure, with steps i) and ii) being as defined above.

In the context of this invention, the ‘percent increase in the number of carbon-centered stable radicals in the bituminous binder’ corresponds to the ratio of the integral value of the ESR signal corresponding to the carbon-centered stable radicals present in the bituminous binder before and after ageing. The value of the integral of the ESR signal is as defined above.

Preferably, the level of susceptibility of the bituminous binder according to the invention is:
extremely low in the case of an increase in the number of stable radicals of less than 40%;
very low in the case of an increase in the number of stable radicals of 40-50%,
low in the case of an increase in the number of stable radicals of 50-60%,
medium in the case of an increase in the number of stable radicals of 60-70%,
medium-high in the case of an increase in the number of stable radicals of 70-80°%,
high in the case of an increase in the number of stable radicals in the number of stable radicals of 80-90%, or
very high in the case of an increase in the number of stable radicals of more than 90%.

This invention also concerns a method for continuous monitoring of the line production of a bituminous binder in a refinery, wherein the continuously produced bituminous binder is analyzed at several points in the production process according to the method specified above.

Preferably, the bituminous binder is analyzed at at least 2 different points.

This monitoring method advantageously allows for the production process of the bituminous binder to be adapted in order to obtain a final bituminous binder with sufficient ageing resistance, and in particular sufficient oxidation resistance.

Preferably, a final bituminous binder having ‘sufficient oxidation resistance’ refers to a bituminous binder having a susceptibility level that was found to be ‘extremely low’, ‘very low’, ‘low’, or ‘medium’ as defined herein.

To this end, the continuous monitoring method according to the invention allows, in particular, for adjusting the proportion of crude oils of different origins involved in the refinement process from which the bituminous binder originates.

This invention also concerns a method for continuous monitoring of the quality of a bituminous binder during its production, wherein the bituminous binder is analyzed at the end of the production process by the method according to the invention.

According to a preferred embodiment, the method for quality control of a bituminous binder according to the invention allows for a determination of whether the addition of an additive to the bituminous binder is required in order to render it more resistant to ageing, and in particular to oxidation. Preferably, the quality control method for a bituminous binder indicates that it is necessary to add an additive when the bituminous binder has a susceptibility level that is ‘medium-high’, ‘high’, or ‘very high’, as defined in this invention.

Preferably, the method for quality control of a bituminous binder allows for a characterization of bituminous binders having very good ageing resistance, in particular bituminous binders having a ‘high’ or ‘very high’ susceptibility to oxidation.

### BRIEF DESCRIPTION OF THE DRAWINGS

FIG. 1 shows an ESR spectrum of a bituminous binder.

FIG. 2 shows the development of the percentage of carbon-centered stable radicals following RTFOT or RTFOT+PAV, normalized relative to the non-aged sample.

### DETAILED DESCRIPTION

The invention will now be described by reference to the following non-limiting examples.

### Ageing of Bituminous Binders

First, the bituminous binder undergoes an RTFOT test, in which a thin film of 80 g of bituminous binder is continuously rotated around the inner surface of a glass bottle at a temperature of 163±0.5° C. The test lasts 75 min, during which hot air is injected every 3-4 s.

This test is considered a realistic simulation of the conditions experienced by a bituminous binder during use, and is governed by European standard EN 12607-1.

The bituminous binder then undergoes a second test, the PAV test, in which 50 g of binder that has undergone the RTFOT test is heated at 100° C. for 20 h at a pressure of 2.1 MPa.

This test allows for a simulation of the ageing of a bituminous binder over a period of 7-10 years of use, and is governed by standard EN 14769.

### Preparation of Samples for ESR Analysis

Prior to the ESR analysis, the aged and non-aged bituminous binders are stored at −20° C. in aluminum receptacles. A sample is taken at a depth of approximately 3 mm using a sharp blade and hand-modelled to obtain a sample of approximately 30 mg, shaped like a grain of rice, 1 cm in length and 3 mm in diameter. The sample is analyzed at 25° C.

### ESR Analysis of Samples

The analyses were conducted in CW (Continuous Wave) mode on a Bruker ELEXSYS-E500 spectrometer. The ESR parameters used are listed in Table 1.

TABLE 1

| Microwave frequency | 9.3 ± 0.1 GHz | |
|---|---|---|
| Microwave power | 0.25 | mW |
| Centre of field | 3500 | G |
| Field amplitude | 150.0 | G |
| Amplitude modulation | 2 | G |
| Modulation frequency | 100 | KHz |
| Time constant | 40.96 | ms |
| Acquisition time | 60 | s |

Table 1. ESR parameters used to analyze the bituminous binder samples.

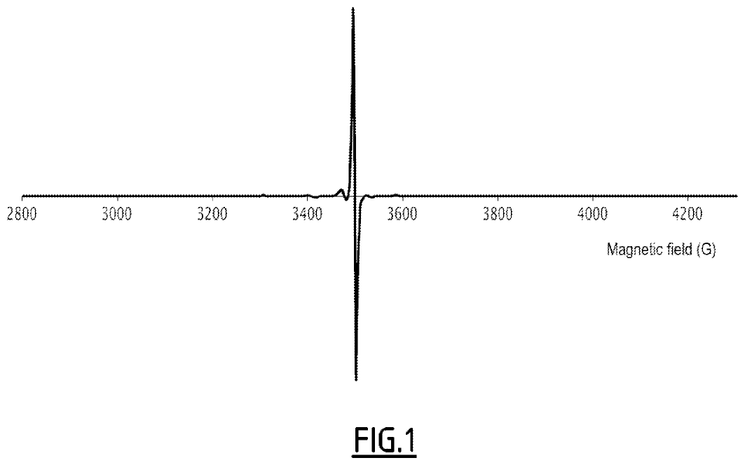

A typical ESR spectrum of a bituminous binder sample is shown in FIG. 1. The majority signal corresponds to the carbon-centered stable radicals, and the minority signals on either side of the majority signal correspond to vanadyl ions.

### Results

Nine samples of bituminous binder were analyzed by the method according to the invention.

The bituminous binders were of various geographic origins (the crudes are Iranian, Venezuelan, Ural, light and heavy Arabian, etc.), all originating from oil refinement, but obtained by different refinement methods:
Y0820-18: 35/50 bitumen from the Feyzin refinery—classic refinement method;
X0490-06: 35/50 bitumen from the Feyzin refinery—classic refinement method;
Y0625-05: 35/50 bitumen from the Donges refinery—classic refinement method;
16-00-756-10: 35/50 bitumen from the Feyzin refinery—classic refinement method;
X1029-03, X1030-05, and X1031-02: 30/45, bitumens of Cuban origin, refinement method unknown;
35/50: corresponds to 35/50 bitumen from the Feyzin refinery, classical method;
70/100 : corresponds to 70/100 bitumen from the Feyzin refinery, obtained by the classical method.

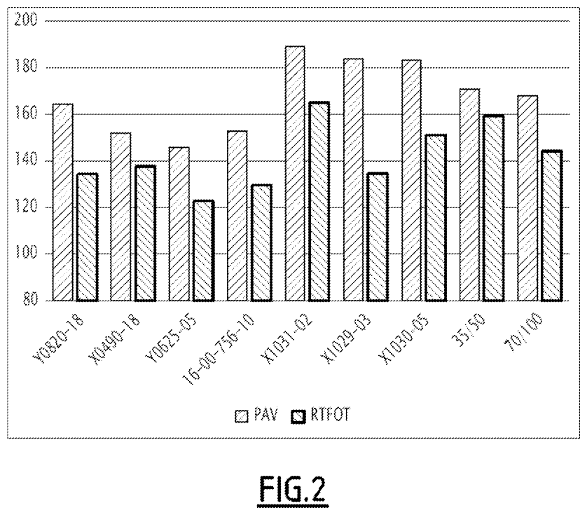

The ESR spectra of these samples were recorded prior to ageing, following the RTFOT test (step i according to the invention), and then following the RTFOT+PAV tests (steps i) and ii) according to the invention). FIG. 2 shows the development of the percentage of carbon-centered stable radicals following each ageing step, normalized relative to the non-aged sample.

The RTFOT and RTFOT+PAV tests both result in an increase in the number of carbon-centered stable radicals. Given that, the greater this increase, the greater the susceptibility of the bituminous binder to oxidation, and thus to ageing, it is possible to establish a classification of the 9 bituminous binders tested based on their ageing resistance. However, this classification differs depending on whether the bituminous binder has undergone the RTFOT test only or the RTFOT+PAV test.

The RTFOT test simulates the conditions experienced by a bituminous binder during initial use, whilst the PAV test allows for a simulation of ageing of a bituminous binder over a period of 7-10 years of use.

Thus, the results obtained on the bitumens that underwent the RTFOT+PAV test allow for a reliable analysis of the long-term ageing stability of a bituminous binder, whilst an ageing method that only involves heating the bituminous binder for a certain amount of time (e.g., the RTFOT test) does not allow for a rapid, reliable analysis of the ageing stability of a bituminous binder.

1. A method for analysing the ageing stability of a bituminous binder, in particular by reference to its susceptibility to oxidation, comprising:
analysing a sample of the bituminous binder by means of electron spin resonance and measuring the integral of the signal of the carbon-centred stable radicals;
accelerated ageing of the bituminous binder, comprising:
i) heating the bituminous binder, followed by
ii) heating the bituminous binder resulting from step i) under pressure;
analysing a sample of the aged bituminous binder obtained from the accelerated ageing step by means of electron spin resonance and measuring the integral of the signal of the carbon-centred stable radicals; and
comparing the integral of the signal of the carbon-centred stable radicals of the bituminous binder obtained from the analysing step and that obtained from the analyzing the aged sample step.

2. The method according to claim 1, wherein step i) of accelerated ageing of the bituminous binder takes place between 100 and 200° C. for a duration of between 5 and 200 min.

3. The method according to claim 1, wherein step ii) of accelerated ageing of the bituminous binder occurs between 50 and 150° C., at a pressure ranging from 0.5-5 MPa, over a duration of between 1 and 100 h.

4. The method according to claim 1, wherein step i) of accelerated ageing of the bituminous binder corresponds to a Rolling Thin Film Over Test.

5. The method according to claim 1, wherein step ii) of accelerated ageing of the bituminous binder corresponds to a long-term accelerated ageing test in a pressure ageing vessel.

6. A method for classifying a bituminous binder as a function of its ageing stability, in particular as a function of its susceptibility to oxidation, comprising:
analysing a sample of the bituminous binder by means of electron spin resonance and measuring the integral of the signal of the carbon-centred stable radicals;
accelerated ageing of the bituminous binder;
analysing a sample of the aged bituminous binder obtained from step 2) by means of electron spin resonance and measuring the integral of the signal of the carbon-centred stable radicals;
determining the percent increase in the number of carbon-centred stable radicals in the bituminous binder by comparing the integral of the signal of the carbon-centred stable radicals of the bituminous binder obtained in the analysing step and that obtained in the analysing the aged sample step; and
determining the level of susceptibility of the bituminous binder:
low in the case of an increase in the number of stable radicals of less than 60%;
medium in the case of an increase in the number of stable radicals of 60-80%, or high in the case of an increase in the number of stable radicals of more than 80%.

7. The method according to claim 6, wherein the accelerated ageing of the bituminous binder comprises: i) heating the bituminous binder, followed by ii) heating the bituminous binder resulting from step i) under pressure.

8. The method according to claim 6, wherein the level of susceptibility of the bituminous binder is:
extremely low in the case of an increase in the number of stable radicals of less than 40%;
very low in the case of an increase in the number of stable radicals of 40-50%,
low in the case of an increase in the number of stable radicals of 50-60%,
medium in the case of an increase in the number of stable radicals of 60-70%,
medium-high in the case of an increase in the number of stable radicals of 70-80%,
high in the case of an increase in the number of stable radicals in the number of stable radicals of 80-90%, or
very high in the case of an increase in the number of stable radicals of more than 90%.

9. A method for continuous monitoring of the production of a bituminous binder, wherein the method is used on a production line in a refinery, wherein the continuously produced bituminous binder is regularly analysed according to the classification method according to claim 6.

10. A method for quality control of a bituminous binder, wherein the bituminous binder is analysed at the end of the production process by the classification method according to claim 6.

11. The method according to claim 7, wherein step i) of accelerated ageing of the bituminous binder takes place between 100 and 200° C. for a duration of between 5 and 200 min.

12. The method according to claim 7, wherein step ii) of accelerated ageing of the bituminous binder occurs between 50 and 150° C., at a pressure ranging from 0.5-5 MPa, over a duration of between 1 and 100 h.

13. The method according to claim 7, wherein step i) of accelerated ageing of the bituminous binder corresponds to a Rolling Thin Film Over Test.

14. The method according to claim 7, wherein step ii) of accelerated ageing of the bituminous binder corresponds to a long-term accelerated ageing test in a pressure ageing vessel.
